(19) United States
(12) Patent Application Publication
(10) Pub. No.: US 2022/0339167 A1
(43) Pub. Date: Oct. 27, 2022

(54) HORMONE D (VITAMIN D) AND ITS DERIVATIVES FOR THE TREATMENT AND PREVENTION OF CANCER

(71) Applicant: Industrial Technologies & Biotechnologies, Playa Blanca-Lanzarote (ES)

(72) Inventor: Victor P. GARCIA, Playa Blanca-Yaiza (ES)

(73) Assignee: Industrial Technologies & Biotechnologies, Playa Blanca-Lanzarote (ES)

(21) Appl. No.: 17/753,087

(22) PCT Filed: Aug. 22, 2019
(86) PCT No.: PCT/IB2019/000787

§ 371 Date: Feb. 18, 2022

(51) Int. Cl. A61K 31/593; A61P 35/00

(52) U.S. Cl. CPC A61K 31/593; A61P 35/00

(57) ABSTRACT

Hormone D (vitamin D) and its derivatives are of the class of secosteroids, compounds derived from a steroid in which there has been a ring cleavage. Humans produce Vitamine D in the skin by photosynthesis, during exposure the sunlight emitting ultraviolet radiation in the narrow band of 290 to 315 nm, from 7-dehydrocholesterol and, consequently, vitamin D is an steroid hormone rather than a true vitamin. 7-Dehydrocholesterol is located in the dermal fibroblast and epidermal keratinocytes. The treatment of several nonmelanoma skin cancers with an oral intake of hormone D₃ (vitamine D₃), at a moderate daily dose resolved all of them completely.

The terminology of vitamin D is highly confusing and even wrong. Humans produce Vitamine D in the skin by photosynthesis, during exposure the sunlight emitting ultraviolet radiation in the narrow band of 290 to 315 nm, from 7-dehydrocholesterol and, consequently, vitamin D is an steroid hormone rather than a true vitamin. 7-Dehydrocholesterol is located in the dermal fibroblast and epidermal keratinocytes.

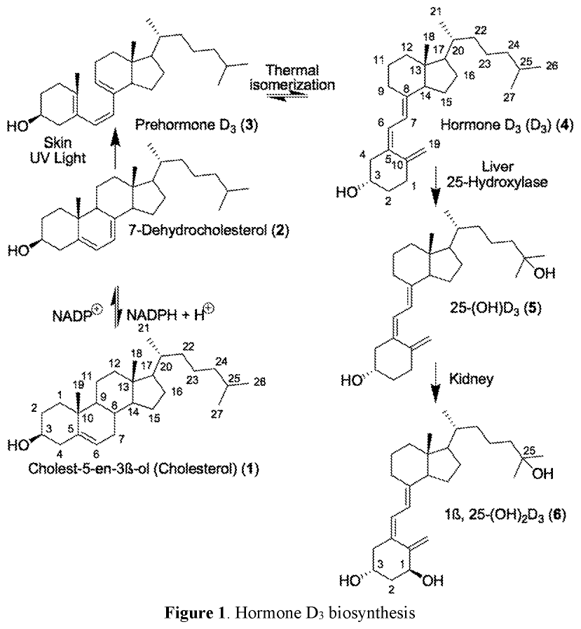

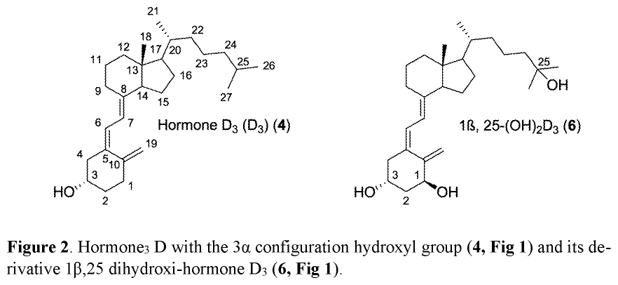

Because aging decreases the capacity of human skin to produce vitamine D those who do not obtain adequate vitamin D from exposure of the skin to environment sunlight must be obtain this essential hormone from their diet (MacLaughlin & Holick, 1985). There are two forms of vitamin D, vitamin D₂ and vitamin D₃, also called ergocalciferol and cholecalciferol, respectively. Vitamin D₂ is derived from irradiation of ergosterol, wich occur to some degree in plankton under natural conditions and is used to produce it from the mold ergot (which contains as much as 2% ergosterol). Vitamin D₂ is manufactured through the ultraviolet irradiation of ergosterol from yeast and fungi (i.e., mushrooms). It is not found in plant material (e.g., vegetables, fruits and grains) and is present in low abundance in meats and other animal food sources except in rare cases such as fish liver oils and the toxic plant waxy-leaf nightshade (Solanum glaucophyllum). Vitamin D₃ is found in fatty fish (i.e., sardine, salmon and mackerel), eggs, and calf liver. Vitamin D₃ is hydroxylated in the liver through the cytochrome P450 enzyme, 25-hydroxylase (CYP2R1) to 25-hydroxyvitamin D₃ (25(OH)D₃), also call calcidiol, the major circulating form of vitamin D. It is hydroxylated to the active metabolite, 1β,25-dihydroxyvitamin D₃ (1,25(OH)₂D₃) or calcitriol in the proximal tubular cells of the kidney. D₃ (1,25(OH)₂D₃) binds to the nuclear vitamin D receptor (VDR) in target organs, then forming heterodimers together with the retinoid X receptor and recruitment other transcriptional cofactors that regulate target gene transcription, including those involved in cell proliferation, differentiation and apoptosis. Finally, another enzyme, 25-hydroxyvitamin D 24-hydroxylase (CYP24A1), inactivates both 25(OH)D₃) and (1,25(OH)₂D₃) respectively to the biologically inactive metabolites 24,25(OH)D₃ and 24,25(OH)₂D₃. Like other hormones, 1β,25-dihydroxyvitamin D₃ circulates at picogram concentrations whereas its precursor circulates at nanograms concentrations. This may be, in part, why 25(OH)D₃, which is also more stable than 1β,25(OH)₂D₃, is currently used to asses clinical vitamin D status, although 1β,25(OH)₂D₃ has much greater affinity for the vitamin D receptor and is more potent and probably the only biologically active form of vitamin D₃. (FIG. 1).

The avian 1α,25(OH)₂D₃ (probably 1 (3,25(OH)₂D₃) has been cloned and shown to be a member of the nuclear transacting receptor family that includes estrogen, progesterone, glucocorticoid, thyrosine (T3), aldosterone, and retinoic acid receptors. The biologically active form 1 β,25(OH)₂D₃ belongs to the steroid family of hormones that share similar mechanisms of action. According to the IUPAC recommendations (Nomenclature of vitamin D. Pure & Appl Chem 54, 8: 1511-16, 1982) forms like 1,25-(OH)₂D₃ is strongly discouraged. We use the term hormone in spite of vitamin, and the terms D₃, 25-Hydroxy D₃ and 1β,25-DihydroxyD₃ despite cholecalciferol (according to IUPAC, the term cholecalciferol may still be used nevertheless calciol but should not be used for naming metabolites), calcidiol and calcitriol, respectively because hormone D₃ has a wide range of functions not only related to calcio metabolism such as cell proliferation, differentiation and apoptosis. Steroid hormones bind to high affinity intracelular receptor (Evans, 1988; Minghetti & Norman, 1988). The biosynthesis of calcitriol is enhance by increasing level of parathyroid hormone (PTH), which rise when the levels of serum calcium or phosphate are lower.

In 1650 Francis Glisson published the first formal medical treatise on rickets. Glisson stated that he had been studying rickets for five years and that it was an “ . . . absolutly new disease, and never described by any ancient or modern writters in their practical books which are extant at this day of the diseases of children. But this disease became first known about 30 years since in the countries of Dorset and Somerset” (Glisson, 1650).

However, the physician Arnold de Boot had published a quite commendable work on rickets in 1649. De Boot was born at Gorcum, in the Netherlands, in 1604. He travelled to London about 1630 to practice medicine and settle in Dublin in 1636 (de Boot, 1649).

In 1822 Sniadecki reported the association of rickets with a lack of sunlight exposure (Sniadecki, 1840). McCollum and colleages published in 1922 that a factor they called vitamin D cure rickets although incorrected named as vitamin because it is an hormone as we said before (McCollum et al., 1922).

Miyaura and colleagues had reported in 1981 that 1α,25-dihydroxyvitamin D₃ [α, 25(OH)₂D₃] (probably 1(3) induces HL-60 differentiation into macrophage-monocyte like cells (Miyaura, 1981). However, as shows the FIG. 1, the configuration of the hydroxy at C-1 of the biologically active metabolite of vitamin D₃ is (3, being a the hydroxy at C-3 position as a consequence of thermal isomeritation, frequent in the chemistry and biochemistry of steroidal compounds. Many reserchers have used modifications of trivial names in an atempt to display relationships between compounds. According to IUPAC, unless otherwise specified, the configuration of the 3-hydroxyl group remain unchanged from that of the 3(3-hydroxyl of the parent tetracyclic steroid. We reported the acid epimerization of 20-keto pregnanglycosides although in a different position (Garcia, 2011). For IUPAC the main confusion in the application of Steroid Rules to vitamin D derivatives is that the description ‘a’ and ‘(3’ only apply when ring A is oriented as in the parent steroid. May be we need the actualization of some rules.

A review of the literature shows that vitamin D₃ has taken a center stage role in our basic and population research quest for the panacea of all human maladies including cancer, yet sufficient evidence for a beneficial role has existed only for skeletal health and osteoporosis prevention singled out by National Academy of Sciences Institute of Medicine Dietary Reference Intake report (Dietary Reference Intakes for Calcium and Vitamin D, 2011). For extraeskeletal outcomes, including cancer, cardiovascular disease, diabetes, and autoinmune disorders the evidence was incosistent, inconclusive as to causality, and insufficient to inform national requirements (Albanes, 2015).

Five studies of serum 25(OH)D₃ in association with colorectal cancer risk were identified in a meta-analysis review using the PubMed database searched for the period from January 1966 to December 2005. All were nested case-control studies who were followed from 2-25 years for incidence. A serum 25(OH)D₃>33 ng/mL (85 nmol/L) was associated with a 50% of colorectal cancer incidence, compared with <12 ng/mL (Gorham et al., 2007)

In the Women's Health Initiative (WHI) trial Wactawski-Wande and colleages found a no significant interaction between the risk of colorectal cancer and supplementation with 1000 mg of elemental calcium as calcium carbonate and 400 IU of vitamin D₃ during an average of seven years of follow-up, probably because of the low dose (Wactawski-Wende et al. 2006).

In 2008, the linking between serum 25(OH)D₃ and the risk of colorectal, breast and prostate cancers and of colorectal adenomas was reviewed by the International Agency for research on Cancer. The results showed evidence for an increased risk of colorectal cancer and colorectal adenoma with low serum 25-hydroxyvitamine D₃ levels, while the evidence for breast cancer was limited, and there was no evidence for prostate cancer (International Agency for research on Cancer, 2008).

A nested case-control study conducted in a population of male Finnish smokers reported that 25(OH)D₃ concentration >65.5 nmol/L (>25.2 ng/mL) was associated with a significante 3-fold increased risk for pancreatic cancer compared whit those with concentrations <32.0 nmol/L (<12.4 ng/mL). The authors reported that this data reflects Findland's northern latitude with less solar UVB photon exposure and less cutaneous vitamin D₃ biosynthesis. Appoximatley 40% of the controls in the study were in the range of 25(OH)D₃ inadequacy (Stolzenberg-Solomon et al., 2006).

In a large, ramdomized multicentre trial conducted in the Prostate, Lung, Colorectal, and Ovarian screening trial cohort of an American population of men and women the authors did not observe a reduced risk between prediagnostic 25(OH)D₃ concentrations, and pancreatic cancer risk. In addition, it has been reported that the highest quintile of 25(OH)D₃ status was associated with a nonsignificant 45% increased pancreatic cancer risk compared with lower 25(OH)D₃ levels. The range of 25(OH)D₃ concentrations in cases was 13.2 to 135.5 nmol/L and in controls was 16.2 to 126.0 nmol/L. In the joint effects models, among subjets with lower 25(OH)D₃ concentrations where positively associated with pancreatic cancer, whereas among subjets with moderate to high residential UBV exposure, 25(OH)D₃ concentrations were non associated with pancreatic cancer (Stolzenberg-Solomon et al., 2009).

The Cohort Consortium Vitamin D Pooling Project of Rarer Cancers showed no evidence for an association between vitamin D₃ status, measure as serum concentrations of 25-hydroxyvitamin D₃ (25 (OH)D₃), and the reduction of less common cancer risk including endometrial, esophageal, gastric, kidney, ovarian, and pancreatic cancers and non-Hodgkin's lymphoma. Moreover, an increased risk at serum levels >40 ng/mL (>100 nmol/L) was reported for pancreatic cancer. A lower risk of upper gastrointestinal cancer has also been observed among Asians individuals in the low range of 25(OH)D₃ (Helzlsouer, 2010).

A significant inverse association between predisgnostic plasma vitamin D₃ and colorectal cancer in women has been reported in a study follow-up for 16 years. The stronghest reduction in incident colorectal cancer and colorectal cancer mortality was observed for 25(OH)D₃ levels greater than 29 ng/mL. The multivariable adjusted model showed that the association was only of borderline significance. However, the baseline plasma 25(OH)D₃ was significantly lower in colorectal cancer cases than in controls (21.9 ng/mL vs 23.9 ng/mL). Limitations of this study include having only a single measure of 25(OH)D₃ and the levels of cholecalciferol (vitamin D₃) and 1β,25(OH)₂D₃, the active form, were not determined (Chandler et al. 2015).

In a large, pooled analysis of men of European ancestry, the authors found for vitamin D₃ genetic variants a direct association with aggresive prostate cancer for six decresing vitamin D₃ categories with median serum 25(OH)D₃ concentration of 65, 61 58, 54, 53, and 43 nmol/L (25.22-16.68 ng/mL), respectively (Mondul et al., 2016), with 40 to 50% higher risk for the highest serum 25(OH)D₃ (Albanes et al., 2011) that appeared stronger in men with higher circulating vitamin D₃ binding protein (DBP) concentrations (Weinstein et al., 2013; Yuan et al., 2018). When they examined prediagnostic serum levels of 25(OH)D₃ and prostate cancer survival found that men with higher serum 25(OH)D₃ were less likely to die from prostate cancer (Mondul et al., 2016).

Oral vitamine D₃, in an initial bolus dose of 200,000 IU, followed by monthly doses of 100,000 IU, or placebo for up to 4 years without calcium has been reported in a randomized clinical trial. The primary outcome of cancer comprised 328 cases of cancer (259 invasive and 69 in situ malignant neoplasems, excludign nonmelanoma skin cancers) and ocurred in 105 of 2558 participants (6.5%) in the vitamin D group and 163 of 2550 (6.4%) in the placebo group. The authors concluded that a high cose vitamin D supplementation prescribes monthly may no prevent cancer (Scrogg et al. 2018).

Manson and coworkers conducted a randomized, placebo-controlled trial fo vitamin D₃ at a dose of 2000 IU dayly and amega-3 fatty acids at a dose of 1 g dayly for the prevention of cancer and cardiovascular disease for 5 years. The authors concluded that dayly supplementation with high-dose vitamin D for 5 years among initially healthy adults in the Unated States not reduce the incidence of cancer or major cardiovascular events (myocardial infarction, stroke, and death form cardiovascular causes) (Manson et al. 2019).

The greatest limitations of these human studies is that vitamin D₃ status was not directly measured. The biologically active form 1β,25(OH)₂D₃ (with 0 configuration at C-1), rather than 1α,25(OH)₂D₃ (with a configuration at C-1), serves as an immunomodulatory hormone and a differentiation hormone besides its clasical role in mineral homeostasis.

The vitamin D₃ receptor (VDR) is a member of the nuclear receptor superfamily and plays a central role in the biological actions of vitamin D (Wang et al., 2012a). VDR affects transcription of nearly 1000 genes because its presence in the target cells of enterocytes (Boos et al., 2007), osteoblasts (Peppel and van Leeuwen, 2014), distal and proximal renal tubule cells, macula densa of the juxtaglomerular apparatus glomerular parietal cells, and podocytes (Wang et al. 2012b). It has been reported the intracellular distribution of the vitamin D receptor in the brain (Prufer et al., 1999). There is abundant evidence for this receptor's presence in the mammalian brain from studies employing immunohistochemistry, western blot analysis or quantitative RNA studies and proteomic techniques (Eyles et al., 2005). VDR is highly expressed in the non-parenchymal cells, Kupffer cells, sinusoidal endothelial cells and specially hepatic stellate cells (Ding et al., 2013), non-malignant, malignant and normal thyroid tissue (Clinskspoor et al., 2012; Clinskspoor & Hauben, 2012), the immune system (promyelocytes, B and T lymphocytes), miocardial cells (Tishop et al. 2008), adipose tissue (for a review see Narvaez C J et al., 2018), bone marrow (Bellido et al., 1993), pituitary gland cells (PerezFernandez et al., 1997), human testis, prostate and in human spermatozoa (Corbet et al., 2006). Hormone D nuclear receptor was detected in parathyroid, pancreatic, pituitary and placental tissues (Pike et al., 1979). VDR mRNA and protein are detected in human endometrium, myometrium, ovarian, cervical and breast tissues (Friedrich et al., 2003; Vienonen et al., 2004). The hormone D receptor has been detected in hair follicle and skin keratinocytes and regulates at least two central process in the skin, interfolicular epidermal differentiation (IFE) and hair follicle cycling (HFC) (Bikle et al., 2015; Bikle, 2015). Hormone D and calcium are well-established regulators of keratinocyte proliferation and differentiation (Bikle, 2015). VDR is also express in cancer cells (Norman, 2006; Sandgran et al., 1991; Lorentzon et al., 2000; d′Alesio et al., 2005).

VDRs have also been reported in the liver (Segura et al., 1999; Garcon Barre et al., 2003), although other groups (Pike et al., 1979; DeLuka et al., 1991) failed to confirm those reports with the use of specific monoclonal antibodies and other methods. However, Han & Chiang have reported the expression of VDR protein and mRNA in HepG2 and human primary hepatocytes. Hepatocytes constitute over 90% of liver mass (Han and Chiang, 2009). It has also been reported a ligand-induced intracellular translocation of VDR from the cytosol to both, the nucleous and plasma membrane, where VDR colocalized with the protein caveolin-1. As in other tissues and cells, VDR has both genomic and nongenomic action in human liver cells. The nongenomic action of membrane VDR signaling is a very rapid response (probably in miliseconds) to cellular stimuli to activate cell-signaling pathways, whereas the genommic action of VDR is a relatively slower response, from minutes to hours, to hormonal ligands by dimerization of VDR with RXR and recruitment of coactivators and/or corepresors to gene promoters to modulate the rate of target gene transcription (Han et al., 2010; Mizwicki et al., 2009).

The few cells or tissues that have either very low or absent VDR expressions include fibroblasts, glomerular mesangial cells, and juxyaglomerular cells (Wang et al., 2012b), interstitial heart (O'Connel & Simpson, 1996; Fraga et al., 2002), red blood cells, such as primitive erythroid progenitors or erythroblasts (Barmincko et al., 2018; Isern et al., 2011), interstitial heart and eskeletal muscle (Bischoff et al., 2001) and smooth muscle (Bouillon et al., 2008; Wang and DeLuka, 2011), and some highly differentiated brain cells, such as the Purkinje cells of the cerebellum (Eyles et al., 2005).

The essential discovery was the identification in many cell types that there is an hormone D₃ receptor within both the nucleous and plasma membrane caveolae, a specialized submicroscopic vesicular organelles, enriched in cholesterol, glicosphingolipids, membrane receptors envolved in cell signaling and membrane transporters, including calcium pumps, that are abundant in many vertebrate cell types. Caveolae were first identified by Palade in 1953 and have now emerged as cell sensors associated with the expresion of caveolins, which work together with coat proteins to regulate the formation of caveolae and the transmission of signals originated in caveolae to several cellular destinations. However, the biophysical characteristics of caveolin, such as its structure, topology, and oligomeric behavier are just biggining to come to light. It has been reported links between caveolae disfunction and human diseases such as muscular dystrophies and cancer (Parton, 2013).

An increasing amount of observations point towards a role for 1,25(OH)₂D₃ signaling in the occurrence and progresion of thyroid cancer, and a potential for structural analogues in the multimodal treatment of dedifferentiated iodine-resist thyroid-cancer (Clickspoor et al., 2013). Altered 1,25(OH)₂D₃-VDR signalling does not influence normal thyroid development nor thyroid function, but does affect c-cell function, at least in rodents.

Thyroid cancer is the most common malignancy of the endocrine system, representing aproximately 1% of all neoplasias. Among them, differentiated thyroid carcinoma (DTC) includes papillary (85% of cases) and follycular (10%) subtypes as the most frequent. It has been reported a higher risk for DTC by haplotypes within the CYP24A1 gene, low circulating 1,25(OH)₂D₃ levels (deficienty), and a reduced conversion to 1,25(OH)₂D₃. VDR, CYP27B1, and CYP24A1 expresion was increased in follicular adenoma (FA) and DTC compared with normal thyroid while in papillary subtype (PTC) with lymph node metastasis, VDR and CYP24A1 were decreasedd compared with non-metastasized PTC. Furthermore, in anaplastic thyroid cancer (ATC), VDR expression was often lost, whereas CYP27B1/CYP24A1 expression was similar to DTC. The authors concluded that there was in increase in the factors related to 1,25(OH)₂D₃ signaling in both non malignante and differentiated malignant thyroid tumors while a decrease was demostrated for local nodal and especially distant metastasis. A streanth of this work was that both 25(OH)D₃ and 1,25(OH)₂D₃ were measured in both patients and controls (Clinskspoor & Hauben, 2012).

The similarity of the action mode of retinoids in relation to the steroid and thyroid hormones has been demostrated with de discovery of the nuclear receptor for retinoic acid, which belongs to the steroid/thyroid hormone receptor superfamily (DeLuka, 1991).

Androgens, which mediate their function by interacting with the intracellualor androgen recptor, play an essential role in many physiological process. Androgen receptor is a member of the superfamily of ligand responsive transcriptional modifiers, which comprises receptors for steroid hormones, such as the hormone D₃ receptor, thyroid hormone receptor and retinoic acid receptor. They all show a similar functional structure (Evans, 1988).

Sun exposure has been associated in ecologic studies with lower death from breast, colorectal, prostate and pancreatic cancer as well as non-Hodgkin's limphoma (Lorentzon et al., 2000; d′Alesio et al., 2005; Wang et al., 2012; Norman, 2006). Furthermore, ecologic studies have shown lower rates of death for cancer and cardiovascular disease in regions with greater sun exposure than in those with less sun exposure (Institute of Medicine, 2011, Manson el al., 2012). However, people of black African descent have vitamin D₃ levels which are below the established range for other populations, despite they do not appear to be vitamin D₃ deficient. Because the endogenous production of vitamin D₃ depends on both latitudinal position and melatonin content, therefore, when skin melatonin content is high, longer periods of sun exposure are necessary for vitamin D₃ biosynthesis (O'Connor et al., 2013; Giovanucci et al., 2006). It has been shown a reduce risk of cancer among normal-weight women and an increase risk in over-weight or obesity compared to the general population under supplementation with hormone D, furthermore, parathyroid hormone (PTH) appears to be suppressed at lower 25-hydroxyhormone D levels in the obese subjets. Shapses and coworkers have shown that the point for near maximal PTH suppression occurred at 21.7 ng/mL and 11.1 ng/mL for the general population and obese women respectively, which would explain hormonal dyregulation related to obesity leading to less benefits of supplementation.

Analogues of 1β,25(OH)₂D₃ inhibit pancreatic cancer cell proliferation, induce differentiation, and promote apoptosis in vitro (Zugmaier et al. 1996; Pettterson et al. 2000; Segura et al., 1999; Fraga et al., 2002). Furthermor, in a ramdomized clinical trial on the effects of sixth months of supplemental calcium (2000 mg/dayly) and vitamin D₃ (800 IU/dayly) results suggest that calcium and vitamin D₃ may enhance apoptosis in normal colonic mucosa base on changes in molecular markers of apoptosis (Golden et al., 2012). Steroids have anti-cancer effects through the induction of apoptosis. In a previous work we reported the cytostatic activities of fuscastatin, a pregnane steroidal compound, against human melanoma SK-MEL-1 cells (data not shown) and human HL-60 cells. Fuscastatin showed cytotoxicity against the human myeloid leukemia cells line HL-60 which was caused by induction of apoptosis as determined by flow cytometry (Garcia et al., 2011). We have also studied the underlying mechanisms of the induction of apoptosis by the cytostatic steroidal compound fuscastatin (unpublished results).

Lithocholic acid (LCA), a highly hydrofobic and toxic bile acid derived from bile acid chenodeoxycholic acid (CDCA) by intestinal bacteria action, is an efficacious endogenous ligand of hormone D receptor (VDR, NR111) (Makishima M, 1996) and pregnane X receptor (NR 112) (Standinger et al., 2001).

We have treated twelve nonmelanoma skin cancers with an oral intake of hormone D₃ (vitamine D₃), at a moderate dayly dose of 466 IU, between 6 and 8 months and all of them were resolved completely.

### REFERENCES

Albanes D, Mondul A M, Yu K, Parisi D, Horst R L, Virtamo J, Weinstein S J. Circulating 25-hydroxyvitamin D and prostate cancer risk in a large neste case-control study. Cancer Epidemiol Biomarkers Prev 2011; 20:1850-60.
Albanes, D. Vitamin D and cancer: diversity, complexity, and still a ways to go. Cancer Prev Res (Phila) 2015; 8:657-61.
Barminko J, Reinholt B M, Emmanuelli A, Lejeuni A N, Baron M H. Activation of the vitamin D receptor transcription factor stimulatres the growth of definitive erythroid progenitors. Blood Adv 2018; 2:1207-19.
Bellido T, Girasole G, Passeri G, Yu X P, Mocharla H, Jilka R L, Notides A, Manolagas S C. Demostration of strogen and vitamin D receptors in bone marrow-derived stromal cells: up-regulation of the estrogen receptor by 1,25-dihydroxyvitamin-D₃. Endocrinology 1993; 133:553-62.
Bikle D D, Oda Y, Tu C L, Jiang Y. Novel mechanisms for the vitamin D receptor (VDR) in the skin and in skin cancer. J Steroid Biochem Mot Blot 2015; 148:47:51.
Bikle D D. Vitamin D receptor, a tumor suppresor in skin. Can J Physiol Pharmacol 2015; 93:349-51.
Bischoff H A, Borchers M, Gudat F, Duermueller U, Theiler R, Stahelin H B, Dick W. In situ detection of 1,25-dihydroxyvitamin D₃ receptor in human skeletal muscle tissue. Histochem J 2001; 33:19-24.
Boos A, Riner K, Hassig M, Liesegang A. Immunohistochemical demonstration of vitamin D receptor distribution in goat intestines. Cells Tissues Organs 2007; 186:121-128.
Bouillon R, Carmeliet G, Verlinden L, van Etten E, Verstuyf A, Luderer H, Lieben L, Mathieu C, Demay M. Vitamin D and human health: lessons from vitamin D receptor null mice. Endocr Rev 2008; 29:726-76.
Chandler P D, Buring J E, Manson J E, Giovannucci E L, Moorthy M V, Zhang S, Lee I M, Lui J H. Circulating vitamin D levels and risk of colorectal cancer in women. Cancer Prev Res 2015; 8:675-82.
Clinskspoor I, Gerard A-C, Saude J V, Many M-C, Verlinden L, Bouillon R, Carmeliet G, Mathieu C, Verstuyf A, Decallonne B. The vitamin D receptor thyroid development and function. Eur Thyroid J 2012; 1:168-75.
Clinskspoor I, Hauben E. Altered expression of key players in vitamin D metabolism and signling in malignant and benign thyroid tumours. J Histochem Cytochem 2012; 60:502-11.
Clinckspoor I, Verlinden L, Mathieu C. Vitamin D in thyroid tumorigenesis and development. Prog Histochem Cytochem 2013; 48:65-98.
Corbett S T, Hill O, Nangia A K. Vitamin D receptor found in human sperm. Urology 2006; 68:1345:49.
d′Alesio A, Garabedian M, Sabatier J P, Guadyer-Souquieres G, Marcelli C, Lemacon A, Walrautr-Debray O, Jehan F. Two single-nucleotide polymorphisems in the human vitamin D receptor prometer change protein DNA complex formation and are associated with hight and vitamin D status in adolescents girls. Hum Mol Genet 2005; 14:3539-48.
de Boot A. Observationes medicae de affectibus a veteribus omissis. London, 1649 DeLuka L M. Retinoids and there receptors in differentiation, embriogenesis, and neoplasia. FASEB 1991; 5:2924-33).
Ding N, Evans R M, Downes M. Hepatic action of vitamine D receptor ligands: a sunshine option for chronic liver disease? Expert Rev Clin Pharmacol 2013; 6(6):597-9.
Evans R M. The steroid and thyroid hormone receptor superfamily. Science 1988; 240:889-95.
Friedrich M, Rafi L, Mitschele T, Tilgen W, Schmidt W, Reichrath J. Analysis of the vitamin D system in cervial carcinomas, breast cancer and ovarian cancer. Recent Results. Cancer Res 2003; 164:239-246.
Eyles D W, Smith S, Kinobe R, Hewison M, McGrath J J. Distribution of the vitamin D receptor and 1α-hydroxylase in human brain. J Chem Neuroanat 2005; 29:21-30.
Fraga C, Blanco M, Vigo E, Segura C, Garcia-Caballero T, Pérez-Fernández R. Ontogenesis of the vitamin D receptor in rat heart. Histochem Cell Biol 2002; 117:547-50.
Garcia V P, Bermejo J, Rubio S, Quintana J, Estévez E. Pregnane steroidal glycosides and their cytostatic activities. Glycobiology 2011; 21; 619-24.
Garcia V P. Acid epimerization of 20-keto pregnanglycosides is determined by 2D-NMR spectroscopy. J Biomol NMR 2011; 50:91-7.
Garcon Barre M, Demers C, Mirshahi A, Neron S, Zaizal S, Nanci A. The normal liver harbours the vitamin D nuclear receptor in nonparenchymal and biliary epithelial cells. Hepatology 2003; 37:1034-42.
Giovanucci E, Lyn Y, Rimm E B, Hollis B W, Fuchs C S, Stampfer M J, Willett W C. Prospective study of predictors of vitamin D status and cancer incidence and mortality in man. J Natl Cancer Inst 2006; 98:451-9.
Glisson F. De rachitide sive morbo puerili, qui vulgó the rickets dicitur, tractatus. London, 1650.
Golden S H, Brown A, Cauley J A, Chin M H, Gary-Webb T L, Kim C, Sosa J A, Summer A E, Anton B. Health disparities in endocrine disorders: biological, clinical, ad nonclinical factors-an endocrine society scientific statement. J Clin Endocrinol Metab 2012; 97:E1579-639.
Gorham E D, Garland C F, Garland F C, Grant W B, Mohr S B, Lipkin M, Newmark H L, Giovannucci E, Wei M, Holick M F. Optimal vitamin D status for colorectal cancer prevention. A quantitative meta analysis. Am J Prev Med. 2007; 32:210-16.
Han S, Chiang J Y. Mechanism of vitamin D receptor inhibition of cholesterol 7α-hydroxylase gene transcription in human hepatocytes. Drug Metab Dispos 2009; 37:469-78.
Han S, Li T, Ellis E, Strom S, Chiang Y S 2010. A novel bile acid-activated vitamin D receptor signaling in human hepatocytes. Mol Endocrinol 24:1151-64.
Helzlsouer K J. Overview of the Cohort Consortium Vitamin D Pooling Project of Rarer Cancers. Am J Epidemiol 2010; 172:4-9.
International Agency for research on Cancer (IARC). Vitamin D and cancer. IARC Working Group Reports Vol 5, International Agency for research on Cancer, Lyon, France, 2008.
Institute of Medicine (TOM). Dietary reference intakes for calcium and vitamin D. The National Academic Press, Washington D.C.: 2011.
Isern J, Fraser S T, Nowotschin S, Ferrer-Vaquer A, Moore R, Hadjantonakis A K, Schulz V, Tuck D, Gallagher P G, Baron M R. Single-lineage transcriptome analysis reveals key regulatory pathways in primitive erythroid progenitors in the mouse embryo. Blood 2011; 117:4924-34.
Lorentzon M, Norstrom P. Vitamin D receptor gene polymorphism is associated with bierth, height, growth to adolescence, and adult stature in healthy caucasian men: a cross-sectional and longitudinal study. J Clin Endocrinol Metab 2000; 85:1666-70.
MacLaughlin J, Holick M. Aging decreases the capacity of human skin to produce vitamin D₃. J Clin Invest 1985; 76:1536-8.
McCollum E V, Simmonds N, Becker J E, Shipley P G. An experimental demonstration of the existence of a vitamin which promotes calcium deposition. J Chem Biol 1922; 53:203-8.
Makishima M, Lu T T, Xie W, Whotfield G K, Domoto H, Evans R M, Hanssler M R, Mangelsdord D J 2002. Vitamin D receptor as an intestinal bile acid sensor. Science 296:1313-16.
Manson J E, Bassuk S S, Lee I M, Cook N R, Albert M A, Gordon D, Azhanis E, Macfadyen J G, Danielson E, Lin J, Zhang S M, Buring J E. The VITamin D and omega-3 trial. (VITAL): rationale and design of a large randomized controlled trial of vitamin D and marine omega-3 fatty acid supplements for the primary prevention of cancer and cardiovascular disease. Comtemp Clin Trials 2012; 33:159-71.
Manson J E, Cook N R, Lee I M, Christen W, Bassuk S S, Mora S, Gibson H, Gordon D, Copeland I, D'Agostino D, Friedenberg G, Ridge C, et al., for the VITAL research group. Vitamin D supplements and prevention of cancer and cardiovascular disease. N Engl J Med 2019; 380:33-44.
Minghetti P P, Norman A W. 1,25(OH)₂-vitamin D₃ receptors: gene regulation and genetic circuitry. FASEB J 1988; 2:3043-53.
Miyaura C, Abe E, Kuribayshi T, Tanaka H, Konno K, Nishii Y, Suda T. 1α,25-dihydroxyvitamin D₃ induces differentiation of human myeloid leukemia cells. Biochem Biophys Res Commun 1981; 102:937-43.
Mizwicki M T, Norman A W 2009. The vitamin D sterol-vitamin D receptor ensemble model offers unique insight into both genomic and rapid response signaling. Sci Signal 2:re4.
Mondul A M, Shui I M, Yu K, Travis C, Stevens V L, Campa D, Schumacher F R, Ziegler R G, Bueno-de-Mesquita H B, Berndt S, Cawford E D, Gapstur S M, Gaziano J M, Giovannucci E, Haiman C A, Henderson B E, Hunter D J, Johansson M, Key T J, Le Marchand L, Lindstroms S, McCullough M L, Navano C, Overvad K, Palli D, Purdue M, Stampfer M J, Weinstein S J, Willett W C, Yeager M, Chacnock S J, Trichopoulos D, Kolonel L N, Kraft P, Albanes D. Genetic variation in the vitamin D pathway in relation to risk of prostate cancer-results from the Breast and Prostate Cancer Cohort Consortium. Cancer Epidemiol Biomarkers Prev 2013; 22:688-96.
Mondul A M, Weinstein S J, Moy K A, Mannisto S, Albanes D. Circulating 25-hydroxyvitamin D and prostate cancer survival. Cancer Epidemiol Biomarkers Prev 2016; 25:665-9.
Narvaez C J, Matthews D G, Welsh J. Vitamin D, vitamin D receptor, and adipose tissue: focus on cellular mechanisms. Vitamin D (fourth edition) 2018:583-596. Nomenclature of vitamin D. Pure & Appl Chem 1982; 54:1511-16.
Norman A W. Minireview: vitamin D receptor: new assigments for an already busy receptor. Endocrinology 2006; 147:5542-8.
O'Connell T, Simpson R U. Immunochemical identification of the 1,25-dihydroxyvitamin D₃ receptor protein in human heart. Cell Bio Int 1996; 20:621-4.
O'Connor M Y, Thoreson, Summers A E. The uncertain significance of low vitamin D levels in African descendent populations: a review of the bone and cardiometabolic literature. Prog Cardiovasc Dis 2013; 56:261-9.
Parton G R, del Pozo M A. Caveolae as plasma membrane sensors, protectors and organizers. Nat Rev Mol Cell Blot 2013; 14:98-112.
Peppel J, Leewen P. Vitamin D gene networks in human osteoblasts. Front Physiol 2014; 5:137.
Pérez-Fernández R, Alonso M, Segura C, Muñoz I, Garcia-Caballero T, Diguez C. Vitamin D receptor gene expression in human pituitary gland. Life Sci 1997; 60:35-42.
Petterson F, Colston K W, Dalgleish A G. Differential and antagonistic effects of 9-cisretinoic acid and vitamin D analogues on pancreatic cancer cells in vitro. Br J Cancer 2000; 83:239-45.
Pike J W, Gooze L L, Haussler M R. Biochemical evidence for 1,25-dihydroxyvitamin D receptor macromolecules in parathyroid, pancreatic pituitary and placental tissues. Life Sci 1979; 26:407-14.
Prufer K, Veenstra T D, Jirikowski G F, Kumar R. Distribution of 1,25-dihydroxyvitamin D₃ receptor immunoreactivity in the rat brain and spinal cord. J Chem Neuroanat 1999; 16:135-45.
Sandgren M E, Bronnegard M, DeLuka H E. Tissue distribution of the 1,25-dihydroxyvitamin D₃ receptor in the male rat. Biochem Biophys Res Comm 1991; 181:611-6.
Segura C, Alonso M, Fraga C, Garcia-Caballero T, Diéguez C, Pérez-Fernández R. Vitamin D receptor ontogenesis in rat liver. Histochem Cell Biol 1999; 112:163-7.
Sniadecki J. Dziela, vol 1, 273-274, Warzawa, 1840.
Standinger J L, Goodwin B, Jones S A, Hawkin-Brown D, Mackenzie K I, LaTour A, Liu Y, Klaasen C D, Brown K K, Reinhard J, Willson T M, Koller H B, Kliever S A. The nuclear receptor PXR is a lithocholic acid sensor that protects against live toxicity. Proc Natl Acad Sci USA 2001; 98:3369-74.
Stolzenberg-Solomon R Z, Vieth R, Azad A, Pietinen P, Taylor P R, Virtamo J, Albanes D. A prospective nested case-control study of vitamin D status and pancreatic cancer risk in male smokers. Cancer Res 2006; 66:10213-9.
Stolzenberg-Solomon R Z, Hayes R B, Horst R L, Anderson K E, Hollis B W, Silverman D T. Serum vitamin D and risk of pancreatic cancer in the Prostate, Lung, Colorectal, and Ovarian screening trial. Cancer Res 2009; 69:1439-47.
Tishkoff D X, Nibbelink K A, Holmberg K H, Dandu L, Simpson R U. Functional vitamin D receptor (VDR) in the T-Tubules of cardiac myocytes: VDR knockout cardiomyocyte contractility. Endocrinology 2008; 149:558-564.
Vienonen A, Miettinen S, Blauer M, Martikainen P M, Tomas E, Heinonen P K, Yikomi T. Expression of nuclear receptors and cofactors in human endometrium and myometrium. J Soc Gynecol Investg 2004; 11:104-112.
Wactawski-Wende J, Kotchen J M, Anderson G L, Assat A R, Brunner R L, Osullivan M J, Margolis K L, Ockene J K, Phillips L, Pottern L, Prenticee R L, Robbins J, Rohan T E, Sarto G E, Sharma S, Stefanick M L, Van Hom L, Wallace R B, Whitlock E, Basford T, Beresford S A, Blaok H R, Bonds D E, Brzyski R G, Caan B, Chlebouski R T, Cochrane B, Garland C, Gass M, Hays J, Hendrix S L, Howard B V, Hsia J, Hubbel F A S, Jackson R D, Johson K C, Judd H, Kooperberg C L, Kuller H I, Lacroix A Z, Lane D S, Langer R D, Laser N L, Lewis C E, Limacher M C, Manson J E; Women's Health Initiative Investigators. Calcium plus vitamin D supplementation and the risk of coloretal cancer N Engl J Med 2006; 354:684-96.
Wang Y, DeLuka H F. Is the vitamin d receptor found in muscle? Endocrinology 2011; 152:354-63.
Wang Y, Zhu J, DeLuca H F. Where is the vitamin D receptor? Arch Biochem Biophys 2012a; 523:123-33.
Wang Y, Borchert M, DeLuka F H, Identification of the vitamin D receptor in various cells of the mouse kidney. Kidney Int 2012b; 81:993-1001.
Weinstein S J, Mondul A M, Kopp W, Rager H, Virtamo J, Albanes D. Circulating 25-hydroxyvitamin D, vitamin D binding protein, and risk of prostate cancer. Int J Cancer 2013; 132:2940-7.
Yuan C, Shui I M, Wilson K M, Stamfer M J, Mucci L A, Giovannucci E L. Circulating 25-hydroxyvitamin D, vitamin D binding protein and risk of advanced and lethal prostate cancer. Int J Cancer 2018; 10.1002/ijc.31966.
Zugmaier D, Jager R, Grage B, Gottardis M M, Havermann K, Knabbe C. Growth-inhibitory effects of vitamin D analogues and retinoids on human pancreatic cancer cells. Br J Cancer 1996; 73:1341-6.

1. The use of an effective amount of cholecalciferol (also called 1β,25-dihydroxyvitamin D₃, 1,25-(OH)₂D₃ or calcitriol) in the manufacture of a pharmaceutical composition for the treatment and prevention of cancer (Structure number 6, FIG. 1).

2. The use of an effective amount of 25-(OH)₂D₃ (also called calcidiol, the major circulating form of vitamin D, which is hydroxylated to the active metabolite, cholecalciferol, in the proximal tubular cells of the kidney) in the manufacture of a pharmaceutical composition for the treatment and prevention of cancer (Structure number 5, FIG. 1), according to claim 1.

3. The use of an effective amount of the compound vitamin D₃ (which is hydroxylated in the liver through the cytochrome P450 enzyme, 25-hydroxylase (CYP2R1) to 25-hydroxyvitamin D₃ or 25-(OH)D₃) in the manufacture of a pharmaceutical composition for the treatment and prevention of cancer (Structure number 4, FIG. 1), according to claim 1.

4. A pharmaceutical composition according to claim 1 for use in the treatment and prevention of nonmelanoma skin cancers.

5. The use of an effective amount of the compound vitamin D₃ (which is hydroxylated in the liver through the cytochrome P450 enzyme, 25-hydroxylase (CYP2R1) to 25-hydroxyvitamin D₃ or 25-(OH)D₃) in the manufacture of a pharmaceutical composition for the treatment and prevention of cancer (Structure number 4, FIG. 1), according to claim 2.

6. A pharmaceutical composition according to claim 2 for use in the treatment and prevention of nonmelanoma skin cancers.

7. A pharmaceutical composition according to claim 3 for use in the treatment and prevention of nonmelanoma skin cancers.

8. A pharmaceutical composition according to claim 5 for use in the treatment and prevention of nonmelanoma skin cancers.
